(19) United States
(12) Patent Application Publication
(10) Pub. No.: US 2005/0288487 A1
(43) Pub. Date: Dec. 29, 2005

(54) Human mast cell-expressed membrane proteins

(76) Inventors: Kang Li, Missouri City, TX (US); Yucheng Li, Houston, TX (US); Shen-Wu Wang, Sugarland, TX (US); Guanghui Hu, Phoenixville, PA (US); Zhengbin Yao, Sugarland, TX (US)

Correspondence Address: Cheryl Liljestrand;Tanox, 10301 Stella Link Road #110, Houston, TX, 77025-5497, US

(21) Appl. No.: 10/500,784

(22) PCT Filed: Jan. 3, 2003
(86) PCT No.: PCT/US03/00307

§ 371 Date: Nov. 3, 2004

Related U.S. Application Data
(60) Provisional application No. 60/345,909, filed on Jan. 3, 2002.

(51) Int. Cl. C12Q 1/68; G01N 33/53; G01N 33/567; C07H 21/04; C07K 14/47

(52) U.S. Cl. 530/350; 435/69.1; 435/320.1; 435/325; 536/23.5; 530/388.1; 435/6; 435/7.2

(57) ABSTRACT

Mast cell-expressed membrane proteins that are highly expressed in human mast cells and the lungs as compared to brain, heart, kidney, liver, tracheal, and similar tissues. The proteins are transmembrane proteins involved in the regulation of mast cell and lung tissue function, including degranulation of mast cells. The preferred protein is a 187 amino acid transmembrane protein having an intercellular domain comprising amino acids 1 through 82, a transmembrane domain comprising amino acids 83 through 105, and an extracellular domain comprising amino acids 106 through 187 (SEQ ID NO:2).

### CROSS REFERENCE TO RELATED APPLICATIONS

This application claims priority to U.S. Provisional Application Ser. No. 60/345,909, filed Jan. 3, 2002, the disclosure of which is incorporated herein by this reference.

### BACKGROUND OF THE INVENTION

1. Field of the Invention

This invention relates generally to cellular membrane proteins and particularly to mast cell-expressed membrane proteins (“MCEMP(s)”).

2. Description of the Prior Art

Mast cells originate from hematopoietic stem cells in the bone marrow but complete their development only after they migrate into diverse peripheral tissues. Mature mast cells express a high-affinity IgE receptor known as FcεRI on their surface. FcεRI can be activated by receptor bound IgE that has been cross-linked with specific allergens. Mast cells can also be activated by IgE independent mechanisms. For example, complement proteins C3a and C5a have been shown to activate mast cells in vivo and calcium ionophores, such as A23187, have been shown to activate mast cells in vitro.

Mast cells contain a wide variety of preformed secretory inflammatory mediators such as histamine, tryptase, proteases, peroxidase, and neutrophil chemotactic factor. Upon activation, mast cells release these preformed mediators and certain newly synthesized lipid mediators such as arachidonic acid metabolites (leukotrienes), prostaglandins, and cytokines into the surrounding tissues. Typically, the cells release both induced immunomodulatory and proinflammatory cytokines, e.g., TNFα, IL4, IL-13, IL-5, IL-10, and chemokines.

It is well known that human mast cells play a critical role in the pathogenesis of many inflammatory and allergic diseases such as asthma and atopic dermatitis. The preformed and newly synthesized mediators released by mast cells are responsible for most of the early events in allergic reactions and, through cytokine production and other mechanisms, contribute to the expression of late-phase reactions and chronic allergic inflammation. Mast cells have also been observed in a multitude of neoplastic, fibrotic, and inflammatory processes such as lymphoproliferative disorders, interstitial lung disease, and the synovium in rheumatoid arthritis. Furthermore, the number of mast cells is highly elevated in other inflammatory diseases such as inflammatory bowel disease. Mast cells also play a role in the progression of heart failure. During heart failure, mast cells are found in the human heart in increased numbers and their density is higher in ischemic cardiomyopathy. U.S. Pat. No. 6,140,348 discloses a method for preventing and treating heart failure by inhibiting mast cell degranulation. Mast cells also play an important role in multiple sclerosis. Mast cell specific genes were found in the brain lesions of multiple sclerosis and a mast cell stabilizer was found to ameliorate the severity of experimental allergic encephalomyelitis (EAE), the animal model of multiple sclerosis.

Since mast cells play such a critical role in allergic and other inflammatory diseases, drugs or other agents that regulate mast cell differentiation, proliferation, adhesion, maturation, activation, and degranulation may be of use to prevent or treat such diseases. Therefore, there is a need to identify novel mast cell proteins that play a role in mast cell mediated diseases and to develop drugs and methods for regulating mast cell activity.

### SUMMARY OF THE INVENTION

It is, therefore, an object of the invention to provide novel mast cell-expressed membrane proteins (“MCEMP(s)”) involved in regulating mast cell activity.

It is another object of the invention to provide agonists or antagonists that bind to MCEMPs and their ligands and regulate their function and activity.

It is another object of the invention to provide antibodies that bind to MCEMPs and methods for producing such antibodies.

It is further object of the invention to provide nucleotide sequences that encode novel MCEMPs.

It is another object of the invention to provide vectors comprising nucleotide sequences that encode novel MCEMPs and host cells containing such vectors.

It is a further object of the invention to provide a screening method for identifying MCEMP agonists and antagonists and for determining whether pharmaceuticals are likely to cause undesirable side effects when administered to an animal.

It is another object of the present invention to provide a method for blocking or modulating the expression of MCEMPs.

It is another object of the present invention to provide a method for diagnosing the predisposition of a mammal to develop diseases caused by the unwanted MCEMP activity.

It is a further object of the invention to provide a method for preventing or treating MCEMP mediated diseases in a mammal.

It is another object of the present invention to provide a diagnostic method for detecting MCEMPs expressed by specific cells, tissues, or body fluids.

It is another object of the present invention to provide a method for isolating and purifying MCEMPs from recombinant cell culture, contaminants, and native environments.

It is a further object of the present invention to provide vaccines and methods for vaccinating a mammal against MCEMP meditated diseases.

These and other objects are achieved by providing a novel MCEMP having the amino sequence shown in SEQ ID NO:2, the nucleotide sequence that codes for the protein, and the vectors and host cells that express the nucleotide sequence and produce the protein. The MCEMP is used to produce agonist and antagonist antibodies useful for affecting mast cell function such as degranulation, adhesion, migration, apoptosis, and the release of mast cell mediators. The antibodies are useful for screening for MCEMP agonists and antagonists and for screening pharmaceuticals to determine if they are likely to cause undesirable side effects when administered to an animal for medicinal purposes.

Other and further objects, features, and advantages of the present invention will be readily apparent to those skilled in the art.

### DETAILED DESCRIPTION OF THE INVENTION

### Definitions

The term “purified polypeptide” means a polypeptide identified and separated from at least one contaminant polypeptide ordinarily associated with the purified polypeptide in its native environment, particularly a polypeptide separated from its cellular environment.

The term “isolated polynucleotide” means a polynucleotide identified and separated from at least one contaminant polynucleotide ordinarily associated with the isolated polynucleotide in its native environment, particularly a polynucleotide separated from its cellular environment.

The term “native” when used to describe a polynucleotide, polypeptide sequence, or other molecule means a polypeptide, polynucleotide, or other molecule as found in nature, e.g., a polypeptide or polynucleotidc sequence that is present in an organism such as a virus or prokaryotic or eukaryotic cell that can be isolated from a source in nature and that has not been intentionally modified to change is structure, properties, or function. An unisolated cellular polynucleotide having the nucleotide sequence shown in SEQ ID NO:1 is a native polynucleotide and unpurified cellular polypeptide having the amino acid sequence shown in SEQ ID NO:2 is a native polypeptide.

The term “percent sequence identity” means the percentage of sequence similarity found in a comparison of two or more nucleotide or amino acid sequences. Percent identity can be determined electronically, e.g., by using the MEGALIGN program (DNASTAR, Inc., Madison Wis.). The MEGALIGN program creates alignments between two or more sequences according to different methods, e.g., the clustal method. (See, e.g., Higgins, D. G. and P. M. Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no similarity between the two amino acid sequences are not included in determining percentage similarity. Percent identity between nucleotide sequences is counted or calculated by methods known in the art, e.g., the Jotun Hein method given in Hein, J. (1990) Methods Enzymol. 183:626-645. Identity between sequences can also be determined by other methods known in the art, e.g., by varying hybridization conditions.

The term “variant” when used to describe a polynucleotide sequence means a nucleotide sequence that differs from its native counterpart by one or more nucleotides and either has the same or similar biological function as its native counterpart or does not have the same or similar biological function as its native counterpart but is useful as a probe to identify or isolate its native counterpart. Preferred variants are nucleotide sequences having at least 85 percent sequence identity when compared to its native counterpart, preferably at least 90 to 95 percent sequence identity, and most preferably at least 99 percent sequence identity, and nucleotide sequences that bind to native sequences or their complement under stringent conditions. Most Preferred variants are nucleotide sequences that code for the same amino acid sequence as its native counterpart but differ from the native nucleotide sequence based only on the degeneracy of the genetic code.

The term “variant” when used to describe a polypeptide sequence means an amino acid sequence that differs from its native counterpart by one or more amino acids, including modifications, substitutions, insertions, and deletions, and either has the same or similar biological function as its native counterpart or does not have the same or similar biological function as its native counterpart but is useful as an immunogen to produce antibodies that bind to its native counterpart or as an agonist or antagonist for its native counterpart. Preferred variants are polypeptides having at least 70 percent sequence identity when compared to its native counterpart, preferably at least 85 percent sequence identity, and most preferably at least 95 percent sequence identity. Most Preferred variants are polypeptides with conservative amino acid substitutions.

The term “fragment” when used to describe a polynucleotide means a nucleotide sequence subset of its native counterpart that binds to its native counterpart or its complement under stringent conditions. Preferred fragments have a nucleotide sequence of at least 25 to 50 consecutive nucleotides of the native sequence. Most preferred fragments have an amino acid sequence of at least 50 to 100 consecutive nucleotides of the native sequence.

The term “fragment” when used to describe a polypeptide means an amino acid sequence subset of its native counterpart that either retains any biological activity of its native counterpart or acts as an immunogen capable of producing an antibody that binds to its native counterpart. Preferred fragments have an amino acid sequence of at least 10 to 20 consecutive amino acids of the native sequence. Most preferred fragments have an amino acid sequence of at least 20 to 30 consecutive amino acids of the native sequence.

The term “agonist” means any molecule that promotes, enhances, or stimulates the normal function of the MCEMPs. One type of agonist is a molecule that interacts with a MCEMP in a way that mimics its ligand, including an antibody or antibody fragment.

The term “antagonist” means any molecule that blocks, prevents, inhibits, or neutralizes the normal function of the MCEMPs. One type of antagonist is a molecule that interferes with the interaction between MCEMPs and its ligand, including an antibody or antibody fragment. Another type of antagonist is an antisense nucleotide that inhibits proper transcription of native MCEMPs.

The term “conservative amino acid substitution” means that an amino acid in a polypeptide has been substituted for with an amino acid having a similar side chain. For example, glycine, alanine, valine, leucine, and isoleucine have aliphatic side chains; serine and threonine have aliphatic-hydroxyl side chains; asparagine and glutamine have amide-containing side chains; phenylalanine, tyrosine, and tryptophan have aromatic side chains; lysine, arginine, and histidine have basic side chains; and cysteine and methionine have sulfur-containing side chains. Preferred conservative amino acids substitutions are valine-leucine-isoleucine, phenylalanine-tyrosine, lysine-arginine, alanine-valine, and asparagine-glutamine.

The term “stringent conditions” means (1) hybridization in 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C., (2) hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C.; with washes at 42° C. in 0.2×SSC and 0.1% SDS or washes with 0.015 M NaCl, 0.0015 M sodium citrate, 0.1% Na₂SO₄ at 50° C. or similar procedures employing similar low ionic strength and high temperature washing agents and similar denaturing agents.

The term “antisense” as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The term “knockout” refers to partial or complete reduction of the expression of at least a portion of a polypeptide encoded by an endogenous gene (such as the gene for MCEMPs) of a single cell, selected cells, or all of the cells of a mammal. The mammal may be a “heterozygous knockout” having one allele of the endogenous gene disrupted or “homozygous knockout” having both alleles of the endogenous gene disrupted.

The term “MCEMP(s)” means amino acid sequences of substantially purified MCEMPs obtained from any species, particularly mammalian, including bovine, ovine, porcine, murine, equine, and preferably human, from any source whether natural, synthetic, semi-synthetic, or recombinant.

This invention is not limited to the particular methodology, protocols, cell lines, vectors, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Because of the degeneracy of the genetic code, a multitude of nucleotide sequences encoding the MCEMPs of the present invention may be produced. Some of these sequences will be highly homologous and some will be minimally homologous to the nucleotide sequences of any known and naturally occurring nucleotide sequence. The present invention contemplates each and every possible variation of nucleotide sequence that could be made by selecting combinations based on possible codon choices. These combinations are made in accordance with the standard triplet genetic code as applied to the nucleotide sequence that codes for naturally occurring MCEMPs and all such variations are to be considered as being specifically disclosed.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents and publications mentioned herein are incorporated herein by reference to the extent allowed by law for the purpose of describing and disclosing the proteins, enzymes, vectors, host cells, and methodologies reported therein that might be used with the present invention. However, nothing herein is to be construed as an admission that the invention is not entitled to antedate such disclosure by virtue of prior invention.

### THE INVENTION

### Polypeptides

In one aspect, the present invention provides a purified polypeptide comprising an amino acid sequence selected from the group consisting of SEQ ID NO:2; a variant of SEQ ID NO:2; a fragment of SEQ ID NO:2; an amino acid sequence encoded by an isolated polynucleotide comprising a nucleotide sequence selected from the group consisting of SEQ ID NO:1; a variant of SEQ ID NO:1; and a fragment of SEQ ID NO:1.

The purified polypeptides of the present invention are mast cell-expressed membrane proteins (“MCEMP(s)”) that are highly expressed in human mast cells and the lungs. The proteins are transmembrane proteins involved in the regulation of mast cell and lung tissue function. In the preferred embodiment, the protein is a 187 amino acid protein having the sequence shown in SEQ ID NO:2 (“MCEMP1”). The preferred protein has an intercellular domain comprising amino acids 1 through 82, a transmembrane domain comprising amino acids 83 through 105, and an extracellular domain comprising amino acids 106 through 187.

The polypeptides of the present invention are used to create antibodies that bind to the proteins and influence mast cell and lung cell structure, properties, or function, including biological functions such as degranulation, adhesion, migration, apoptosis, and the release of mast cell contents. Preferably, the antibodies function as MCEMP agonists to activate the production of mast cell mediators or as MCEMP antagonists to inhibit the production of mast cell proinflammnatory mediators such as histamines, TNFα, and leukotrienes.

### Agonists and Antagonists

In another aspect, the present invention provides agonists and antagonists that specifically bind to a MCEMP or its ligand and inhibit or activate its cellular function. Types of agonist and antagonists include, but are not limited to, polypeptides, proteins, peptides, glycoproteins, glycopeptides, glycolipids, polysaccharides, oligosaccharides, nucleotides, organic molecules, bioorganic molecules, peptidomimetics, pharmacological agents and their metabolites, and transcriptional and translation control sequences.

In one embodiment, the antagonists are a soluble form of MCEMP and soluble polypeptides derived from the extracellular domains of MCEMPs that are capable of interfering with the ability of a MCEMP to interact with its natural ligand. Preferably, the antagonists are peptides selected from the group consisting of amino acids 106 through 187 of SEQ ID NO:2 or antagonist fragments thereof. These antagonistic block the binding of the natural ligand for MCEMPs by binding to the ligand and preventing the ligand from binding to its native receptor.

Preferably, the agonists and antagonists are antibodies that bind specifically to MCEMP and influence their biological actions and functions, e.g., to activate or inhibit degranulation and control the release of mast cell mediators. The antibodies can be polyclonal or monoclonal antibodies but are preferably monoclonal antibodies.

Antagonist antibodies are used to prevent or treat diseases characterized by the activation of mast cells, e.g., diseases caused by degranulation and the release of mast cell contents. Agonist antibodies are used to prevent or treat diseases characterized by relatively low mast cell mediator concentration.

The agonists and antagonists are used for the treatment of various immune diseases, including, but not limited to allergic diseases such as asthma, allergic rhinitis, atopic dermatitis, food hypersensitivity and urticaria; transplantation associated diseases including graft rejection and graft-versus-host-disease; autoimmune or immune-mediated skin diseases including bullous skin diseases, erythema multiforme and contact dermatitis, psoriasis; rheumatoid arthritis, juvenile chronic arthritis; inflammatory bowel disease (i.e., ulcerative colitis, Crohn's disease); systemic lupus erythematosis; spondyloarthropathies; systemic sclerosis (seleroderma); idiopathic inflammatory myopathies (dermatomyositis, polymyositis); Sjogren's syndrome; systemic vasculitis; sarcoidosis; autoimmune hemolytic anemia (immune pancytopenia, paroxysmal nocturnal hemoglobinuria), autoimmune thrombocytopenia (idiopathic thrombocytopenic purpura, immune-mediated thrombocytopenia); thyroiditis (Grave's disease, Hashimoto's thyroiditis, juvenile lymphocytic thyroiditis, atrophic thyroiditis); diabetes mellitus; immune-mediated renal disease (glomerulonephritis, tubulointerstitial nephritis); demyelinating diseases of the central and peripheral nervous systems such as multiple sclerosis, idiopathic demyelinatingpolyneuropathy or Guillain-Barre syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis; inflammatory and fibrotic lung diseases such as cystic fibrosis, gluten-sensitive enteropathy, and Whipple's disease; immunologic diseases of the lung such as eosinophilic pneumonia, idiopathic pulmonary fibrosis and hypersensitivity pneumonitis.

### Antibody and Antibody Production

In another aspect, the present invention provides an antibody that binds to the MCEMPs of the present invention and methods for producing such antibody, including antibodies that function as MCEMP agonists or antagonists. In one embodiment, the method comprises using isolated MCEMPs or antigenic fragments thereof as an antigen for producing antibodies that bind to the MCEMPs of the present invention in a known protocol for producing antibodies to antigens, including polyclonal and monoclonal antibodies. In another embodiment, the method comprises using host cells that express recombinant MCEMPs as an antigen. In a further embodiment, the method comprises using DNA expression vectors containing the MCEMP gene to express the MCEMP as an antigen for producing the antibodies.

Methods for producing antibodies, including polyclonal, monoclonal, monovalent, humanized, human, bispecific, and heteroconjugate antibodies, are well known to skilled artisans.

### Polyclonal Antibodies

Polyclonal antibodies can be produced in a mammal by injecting an immunogen alone or in combination with an adjuvant. Typically, the immunogen is injected in the mammal using one or more subcutaneous or intraperitoneal injections. The immunogen may include the polypeptide of interest or a fusion protein comprising the polypeptide and another polypeptide known to be immunogenic in the mammal being immunized. The immunogen may also include cells expressing a recombinant MCEMP or a DNA expression vector containing the MCEMP gene. Examples of such immunogenic proteins include, but are not limited to, keyhole limpet hemocyanin, serum albumin, bovine thyroglobulin, and soybean trypsin inhibitor. Examples of adjuvants include, but are not limited to, Freund's complete adjuvant and MPL-TDM adjuvant (monophosphoryl Lipid A, synthetic trehalose dicorynomycolate). The immunization protocol may be selected by one skilled in the art without undue experimentation.

### Monoclonal Antibodies

Monoclonal antibodies can be produced using hybridoma methods such as those described by Kohler and Milstein, Nature, 256:495 (1975). In a hybridoma method, a mouse, hamster, or other appropriate host mammal, is immunized with an immunogen to elicit lymphocytes that produce or are capable of producing antibodies that will specifically bind to the immunogen. Alternatively, the lymphocytes may be immunized in vitro. The immunogen will typically include the polypeptide of interest or a fusion protein containing such polypeptide. Generally, peripheral blood lymphocytes (“PBLs”) cells are used if cells of human origin are desired. Spleen cells or lymph node cells are used if cells of non-human mammalian origin are desired. The lymphocytes are then fused with an immortalized cell line using a suitable fusing agent, e.g., polyethylene glycol, to form a hybridoma cell (Goding, Monoclonal Antibodies: Principles and Practice, pp 59-103 (Academic Press, 1986)). Immortalized cell lines are usually transformed mammalian cells, particularly rodent, bovine, or human myeloma cells. Usually, rat or mouse myeloma cell lines are employed. The hybridoma cells may be cultured in a suitable culture medium that preferably contains one or more substances that inhibit the growth or survival of the unfused immortalized cells. For example, if the parental cells lack the enzyme hypoxanthine guanine phosphoribosyl transferase (HGPRT), the culture medium for the hybridomas typically will include hypoxanthine, aminopterin, and thymidine (HAT medium). The HAT medium prevents the growth of HGPRT deficient cells.

Preferred immortalized cell lines are those that fuse efficiently, support stable high level expression of antibody by the selected antibody producing cells, and are sensitive to a medium such as HAT medium. More preferred immortalized cell lines are murine myeloma lines such as those derived from MOPC-21 and MPC-11 mouse tumors available from the Salk Institute Cell Distribution Center, San Diego, Calif. USA, and SP2/0 or X63-Ag8-653 cells available from the American Type Culture Collection, Rockville, Md. USA. Human myeloma and mouse-human heteromyeloma cell lines also have been described for use in the production of human monoclonal antibodies (Kozbor, J. lmmunol. 133:3001 (1984); Brodeur et al., Monoclonal Antibody Production Techniques and Applications, pp. 51-63 (Marcel Dekker, Inc., New York, 1987)). The mouse myeloma cell line NS0 may also be used (European Collection of Cell Cultures, Salisbury, Wiltshire UK). Human myeloma and mouse-human heteromycloma cell lines, well known in the art, can also be used to produce human monoclonal antibodies.

The culture medium used for culturing hybridoma cells can then be assayed for the presence of monoclonal antibodies directed against the polypeptide of interest. Preferably, the binding specificity of monoclonal antibodies produced by the hybridoma cells is determined by immunoprecipitation or by an in vitro binding assay, e.g., radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem, 107:220 (1980).

After the desired hybridoma cells are identified, the clones may be subcloned by limiting dilution procedures and grown by standard methods. Suitable culture media for this purpose include Dulbecco's Modified Eagle's Medium and RPMI-1640 medium. Alternatively, the hybridoma cells may be grown in vivo as ascites in a mammal.

The monoclonal antibodies secreted by the subclones are isolated or purified from the culture medium or ascites fluid by conventional immunoglobulin purification procedures such as protein A-Sepharose, hydroxylapatite chromatography, gel electrophoresis, dialysis, or affinity chromatography.

The monoclonal antibodies may also be produced by recombinant DNA methods, e.g., those described in U.S. Pat. No. 4,816,567. DNA encoding the monoclonal antibodies of the invention can be readily isolated and sequenced using conventional procedures, e.g., by using oligonucleotide probes that are capable of binding specifically to genes encoding the heavy and light chains of murine antibodies (Innis M. et al. In “PCR Protocols. A Guide to Methods and Applications”, Academic, San Diego, Calif. (1990), Sanger, F. S, et al. Proc. Nat. Acad. Sci. 74:5463-5467 (1977)). The hybridoma cells described herein serve as a preferred source of such DNA. Once isolated, the DNA may be placed into expression vectors. The vectors are then transfected into host cells such as simian COS cells, Chinese hamster ovary (CHO) cells, or myeloma cells that do not otherwise produce immunoglobulin protein. The recombinant host cells arc used to produce the desired monoclonal antibodies. The DNA also may be modified, for example, by substituting the coding sequence for human heavy and light chain constant domains in place of the homologous murine sequences or by covalently joining the immunoglobulin coding sequence to all or part of the coding sequence for a non-immunoglobulin polypeptide. Such a non-immunoglobulin polypeptide can be substituted for the constant domains of an antibody or can be substituted for the variable domains of one antigen combining site of an antibody to create a chimeric bivalent antibody.

Monovalent antibodies can be produced using the recombinant expression of an immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking. Similarly, in vitro methods can be used for producing monovalent antibodies. Antibody digestion can be used to produce antibody fragments, preferably Fab fragments, using known methods.

Antibodies and antibody fragments can be produced using antibody phage libraries generated using the techniques described in McCafferty, et al., Nature 348:552-554 (1990). Clackson, et al., Nature 352:624-628 (1991) and Marks, et al., J. Mol. Biol. 222:581-597 (1991) describe the isolation of murine and human antibodies, respectively, using phage libraries. Subsequent publications describe the production of high affinity (nM range) human antibodies by chain shuffling (Marks, et al., Bio/Technology 10:779-783 (1992)), as well as combinatorial infection and in vivo recombination as a strategy for constructing very large phage libraries (Waterhouse, et al., Nuc. Acids. Res. 21:2265-2266 (1993)). Thus, these techniques are viable alternatives to traditional monoclonal antibody hybridoma techniques for isolation of monoclonal antibodies. Also, the DNA may be modified, for example, by substituting the coding sequence for human heavy-chain and light-chain constant domains in place of the homologous murine sequences (U.S. Pat. No. 4,816,567; Morrison, et al., Proc. Nat. Acad. Sci. USA 81:6851 (1984)), or by covalently joining to the immunoglobulin coding sequence all or part of the coding sequence for a non-immunoglobulin polypeptide. Typically, such ion-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Antibodies can also be produced using use electrical fusion rather than chemical fusion to form hybridomas. This technique is well established. Instead of fusion, one can also transform a B-cell to make it immortal using, for example, an Epstein Barr Virus, or a transforming gene “Continuously Proliferating Human Cell Lines Synthesizing Antibody of Predetermined Specificity,” Zurawaki, V. R. et al, in “Monoclonal Antibodies,” ed. by Kennett R. H. et al, Plenum Press, N.Y. 1980, pp 19-33.

### Humanized Antibodies

Humanized antibodies can be produced using the method described by Winter in Jones et al., Nature, 321:522-525 (1986); Riechmann et al., Nature, 332:323-327 (1988); and Verhoeyen et al., Science, 239:1 534-1536 (1988). Humanization is accomplished by substituting rodent CDRs or CDR sequences for the corresponding sequences of a human antibody. Generally, a humanized antibody has one or more amino acids introduced into it from a source that is non-human. Such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Humanized forms of non-human (e.g., murine or bovine) antibodies are chimeric immunoglobulins, immunoglobulin chains, or immunoglobulin fragments such as Fv, Fab, Fab′, F(ab′)₂, or other antigen-binding subsequences of antibodies that contain minimal sequence derived from non-human immunoglobulin. Humanized antibodies include human immunoglobulins (recipient antibody) wherein residues from a complementary determining region (CDR) of the recipient are replaced by residues from a CDR of a non-human species (donor antibody) such as mouse, rat, or rabbit having the desired specificity, affinity, and capacity. Sometimes, Fv framework residues of the human immunoglobulin are replaced by corresponding non-human residues. Humanized antibodies also comprise residues that are found neither in the recipient antibody nor in the imported CDR or framework sequences. In general, humanized antibodies comprise substantially all of at least one and typically two variable domains wherein all or substantially all of the CDR regions correspond to those of a non-human immunoglobulin and all or substantially all of the FR regions are those of a human immunoglobulin consensus sequence. Humanized antibodies optimally comprise at least a portion of an immunoglobulin constant region (Fc), typically that of a human immunoglobulin.

### Human Antibodies

Human antibodies can be produced using various techniques known in the art, e.g., phage display libraries as described in Hoogenboom and Winter, J. Mol. Biol., 227:381 (1991) and Marks et al., J. Mol. Biol., 222:581 (1991). Human monoclonal antibodies can be produced using the techniques described in Cole et al., Monoclonal Antibodies and Cancer Therapy, Alan R. Liss, p. 77 (1985) and Boemer et al., J. Immunol., 147(1):86-95 (1991). Alternatively, transgenic animals, e.g., mice, are available which, upon immunization, can produce a full repertoire of human antibodies in the absence of endogenous immunoglobulin production. Such transgenic mice are available from Abgenix, Inc., Fremont, Calif., and Medarex, Inc., Annandale, N.J. It has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA 90:2551 (1993); Jakobovits et al., Nature 362:255-258 (1993); Bruggermann et al., Year in Immunol. 7:33 (1993); and Duchosal et al. Nature 355:258 (1992). Human antibodies can also be derived from phage-display libraries (Hoogenboom et al., J. Mol. Biol. 227:381 (1991); Marks et al., J. Mol. Biol. 222:581-597 (1991); Vaughan et al., Nature Biotech 14:309 (1996)).

### Bispecific Antibodies

Bispecific antibodies can be produced by the recombinant co-expression of two immunoglobulin heavy-chain/light-chain pairs wherein the two heavy chains have different specificities. Bispecific antibodies are monoclonal, preferably human or humanized, antibodies that have binding specificities for at least two different antigens. In the present invention, one of the binding specificities is for the MCEMP and the other is for any other antigen, preferably a cell surface receptor or receptor subunit. Because of the random assortment of immunoglobulin heavy and light chains, these hybridomas produce a potential mixture of ten different antibodies. However, only one of these antibodies has the correct bispecific structure. The recovery and purification of the correct molecule is usually accomplished by affinity chromatography.

Antibody variable domains with the desired binding specificities (antibody-antigen combining sites) can be fused to immunoglobulin constant domain sequences. The fusion preferably is with an immunoglobulin heavy chain constant domain comprising at least part of the hinge, CH2, and CH3 regions. Preferably, the first heavy-chain constant region (CH1) containing the site necessary for light-chain binding is present in at least one of the fusions. DNAs encoding the immunoglobulin heavy-chain and, if desired, the immunoglobulin light chain is inserted into separate expression vectors and co-transfected into a suitable host organism. Suitable techniques are shown in for producing bispecific antibodies are described in Suresh et al., Methods in Enzymology, 121:210 (1986).

### Heteroconjugate Antibodies

Heteroconjugate antibodies can be produced known protein fusion methods, e.g., by coupling the amine group of one an antibody to a thiol group on another antibody or other polypeptide. If required, a thiol group can be introduced using known methods. For example, immunotoxins comprising an antibody or antibody fragment and a polypeptide toxin can be produced using a disulfide exchange reaction or by forming a thioether bond. Examples of suitable reagents for this purpose include iminothiolate and methyl-4-mercaptobutyrimidate. Such antibodies can be used to target immune system cells to unwanted cells or to treat HIV infections.

### Polynucleotides

In another aspect, the present invention provides an isolated polynucleotide comprising a nucleotide sequence selected from the group consisting of SEQ ID NO:1; a variant of SEQ ID NO:1; a fragment of SEQ ID NO:1; a nucleotide sequence that encodes a polypeptide having the amino acid sequence selected from the group consisting of SEQ ID NO:2; a variant of SEQ ID NO:2; and a fragment of SEQ ID NO:2. In one embodiment, the isolated polynucleotide comprises a nucleotide sequence that encodes a polypeptide having an amino acid sequence selected from the group consisting of amino acids 106 to 187 of SEQ ID NO:2 or antagonist fragments thereof.

The isolated polynucleotides of the present invention are preferably coding sequences for MCEMPs involved in the regulation of mast cell and lung function. The polynucleotides are used to produce MCEMPs that function as antigens in the process used to produce the agonist and antagonist antibodies that specifically bind to MCEMPs and inhibit or activate the degranulation of mast cells.

### Vectors and Host Cells

In another aspect, the present invention provides a vector comprising a nucleotide sequence encoding the MCEMPs of the present invention and a host cell comprising such a vector. The vector may contain SEQ ID NO: 2 or, in one embodiment, nucleotides 455 through 1018 of SEQ ID NO:1 in combination with any regulatory, expression, or other vector sequences required to express MCEMPs.

By way of example, the host cells may be mammalian cells, (e.g. CHO cells), prokaryotic cells (e.g., E. coli) or yeast cells (e.g., Saccharomyces cerevisiae). A process for producing vertebrate fused polypeptides is further provided and comprises culturing host cells under conditions suitable for expression of vertebrate fused and recovering the same from the cell culture. The present invention includes the proteins and polypeptides with or without associated native-pattern glycosylation. The recombinant proteins when expressed in yeast or mammalian expression systems (e.g., COS-7 cells) may be similar or significantly different in molecular weight and glycosylation pattern from the corresponding native proteins. Expression of mammalian MCEMPs in bacterial expression systems, such as E. coli, provides non-glycosylated molecules. Variant proteins comprising inactivated N-glycosylation sites are also within the scope of the present invention. Such variants are expressed in a more homogeneous, reduced carbohydrate form.

### Recombinant Expression for MCEMPs

Isolated and purified recombinant MCEMPs are provided according to the present invention by incorporating the corresponding nucleotide sequence into expression vectors and expressing the nucleotide sequence in suitable host cells to produce the polypeptide.

### Expression Vectors

Recombinant expression vectors containing a nucleotide sequence encoding the polypeptide can be prepared using well known techniques. The expression vectors include a nucleotide sequence operably linked to suitable transcriptional or translational regulatory nucleotide sequences such as those derived from mammalian, microbial, viral, or insect genes. Examples of regulatory sequences include transcriptional promoters, operators, enhancers, mRNA ribosomnal binding sites, and appropriate sequences which control transcription and translation initiation and termination. Nucleotide sequences are “operably linked” when the regulatory sequence functionally relates to the nucleotide sequence for the appropriate polypeptide. Thus, a promoter nucleotide sequence is operably linked to a MCEMP sequence if the promoter nucleotide sequence controls the transcription of the appropriate nucleotide sequence.

The ability to replicate in the desired host cells, usually conferred by an origin of replication and a selection gene by which transformants are identified, may additionally be incorporated into the expression vector.

In addition, sequences encoding appropriate signal peptides that are not naturally associated with MCEMPs can be incorporated into expression vectors. For example, a nucleotide sequence for a signal peptide (secretory leader) may be fused in-frame to the polypeptide sequence so that the polypeptide is initially translated as a fusion protein comprising the signal peptide. A signal peptide that is functional in the intended host cells enhances extracellular secretion of the appropriate polypeptide. The signal peptide may be cleaved from the polypeptide upon secretion of polypeptide from the cell.

### Host Cells

Suitable host cells for expression of MCEMPs include prokaryotes, yeast, archae, and other eukaryotic cells. Appropriate cloning and expression vectors for use with bacterial, fungal, yeast, and mammnalian cellular hosts are well known in the art, e.g., Pouwels et al. Cloning Vectors: A Laboratory Manual, Elsevier, N.Y. (1985). The vector may be a plasmid vector, a single or double-stranded phage vector, or a single or double-stranded RNA or DNA viral vector. Such vectors may be introduced into cells as polynucleotides, preferably DNA, by well known techniques for introducing DNA and RNA into cells. The vectors, in the case of phage and viral vectors also may be and preferably are introduced into cells as packaged or encapsulated virus by well known techniques for infection and transduction. Viral vectors may be replication competent or replication defective. In the latter case viral propagation generally will occur only in complementing host cells. Cell-free translation systems could also be employed to produce the protein using RNAs derived from the present DNA constructs.

Prokaryotes useful as host cells in the present invention include gram negative or gram positive organisms such as E. coli or Bacilli. In a prokaryotic host cell, a polypeptide may include a N-terminal methionine residue to facilitate expression of the recombinant polypeptide in the prokaryotic host cell. The N-terminal Met may be cleaved from the expressed recombinant MCEMPs. Promoter sequences commonly used for recombinant prokaryotic host cell expression vectors include β-lactamase and the lactose promoter system.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. To construct an expression vector using pBR322, an appropriate promoter and a DNA sequence are inserted into the pBR322 vector. Other commercially available vectors include, for example, pKK223-3 (Pharmacia Fine Chemicals, Uppsala, Sweden), pGEM1 (Promega Biotec, Madison, Wis., USA), and the pET (Novagen, Madison, Wis., USA) and pRSET (Invitrogen Corporation, Carlsbad, Calif., USA) series of vectors (Studier, F. W., J. Mol. Biol. 219: 37 (1991); Schoepfer, R. Gene 124: 83 (1993)).

Promoter sequences commonly used for recombinant prokaryotic host cell expression vectors include T7, (Rosenberg, A. H., Lade, B. N., Chui, D-S., Lin, S-W., Dunn, J. J., and Studier, F. W. (1987) Gene (Amst.) 56, 125-135), β-lactamasc (penicillinase), lactose promoter system (Chang et al., Nature 275:615, (1978); and Goeddel et al., Nature 281:544, (1979)), tryotophan (trp) promoter system (Goeddel et al., Nucl. Acids Res. 8:4057, (1980)), and tac promoter (Maniatis, Molecular Cloning: A Laboratory Manual, Cold Spring Harbor Laboratory, p.412 (1982)).

Yeasts useful as host cells in the present invention include those from the genus Saccharomyces, Pichia, K. Actinomycetes and Kluyveromyces. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991). Other suitable promoters and vectors for yeast and yeast transformation protocols are well known in the art.

Yeast transformation protocols are known to those of skill in the art. One such protocol is described by Hinnen et al., Proceedings of the National Academy of Sciences USA, 75:1929 (1978). The Hinnen protocol selects for Trp.sup.+transformants in a selective medium, wherein the selective medium consists of 0.67% yeast nitrogen base, 0.5% casamino acids, 2% glucose, 10 μg/ml adenine, and 20 μg/ml uracil.

Mammalian or insect host cell culture systems well known in the art could also be employed to express recombinant MCEMPs, e.g., Baculovirus systems for production of heterologous proteins in insect cells (Luckow and Summers, Bio/Technology 6:47 (1988)) or Chinese hamster ovary (CHO) cells for mammalian expression may be used. Transcriptional and translational control sequences for mammalian host cell expression vectors may be excised from viral genomes. Commonly used promoter sequences and enhancer sequences are derived from Polyoma virus, Adenovirus 2, Simian Virus 40 (SV40), and human cytomegalovirus. DNA sequences derived from the SV40 viral genome may be used to provide other genetic elements for expression of a structural gene sequence in a mammalian host cell, e.g., SV40 origin, early and late promoter, enhancer, splice, and polyadenylation sites. Viral early and late promoters are particularly useful because both are easily obtained from a viral genome as a fragment which may also contain a viral origin of replication. Exemplary expression vectors for use in mammalian host cells are well known in the art.

MCEMPs may, when beneficial, be expressed as a fusion protein that has the MCEMP attached to a fusion segment. The fusion segment often aids in protein purification, e.g., by permitting the fusion protein to be isolated and purified by affinity chromatography. Fusion proteins can be produced by culturing a recombinant cell transformed with a fusion nucleic acid sequence that encodes a protein including the fusion segment attached to either the carboxyl and/or amino terminal end of the protein. Preferred fusion segments include, but are not limited to, glutathione-S-transferase, β-galactosidase, a poly-histidine segment capable of binding to a divalent metal ion, and maltose binding protein.

Since the MCEMPs lack a discernable leader peptide, a heterologous signal peptide may be advantageously fused to the N-terminus of a soluble MCEMP to promote secretion thereof. The signal peptide can be cleaved from the protein upon secretion from the host cell. The need to lyse the cells and recover the recombinant soluble protein from the cytoplasm thus is avoided. In one embodiment of the invention, a soluble fusion protein comprises a first polypeptide derived from the extracellular domain of MCEMP1 fused to a second polypeptide added for purposes such as facilitating purification or effecting dimer formation. Suitable second polypeptides do not inhibit secretion of the soluble fusion protein. Examples of soluble polypeptides include those comprising the entire extracellular domain. Representative examples of the soluble proteins of the present invention include, but are not limited to, a polypeptide comprising amino acids of SEQ ID NO:2, wherein the polypeptide is selected from amino acids 1 through 82 of SEQ ID NO:2, amino acids 6 through 65 of SEQ ID NO:2, or any fragment thereof that retains the ability to bind MCEMP1 ligand. Truncated forms of the inventive proteins, including soluble polypeptides, may be prepared by any of a number of conventional techniques.

### Expression and Recovery

According to the present invention, isolated and purified MCEMPs may be produced by the recombinant expression systems described above. The method comprises culturing a host cell transformed with an expression vector comprising a nucleotide sequence that encodes the polypeptide under conditions sufficient to promote expression of the polypeptide. The polypeptide is then recovered from culture medium or cell extracts, depending upon the expression system employed. As is known to the skilled artisan, procedures for purifying a recombinant polypeptide will vary according to such factors as the type of host cells employed and whether or not the recombinant polypeptide is secreted into the culture medium. When expression systems that secrete the recombinant polypeptide are employed, the culture medium first may be concentrated. Following the concentration step, the concentrate can be applied to a purification matrix such as a gel filtration medium. Alternatively, an anion exchange resin can be employed, e.g., a matrix or substrate having pendant diethylaminoethyl (DEAE) groups. The matrices can be acrylamide, agarose, dextran, cellulose, or other types commonly employed in protein purification. Also, a cation exchange step can be employed. Suitable cation exchangers include various insoluble matrices comprising sulfopropyl or carboxymethyl groups. Further, one or more reversed-phase high performance liquid chromatography (RP-HPLC) steps employing hydrophobic RP-HPLC media (e.g., silica gel having pendant methyl or other aliphatic groups), ion cxchange-HPLC (e.g., silica gel having pendant DEAE or sulfopropyl (SP) groups), or hydrophobic interaction-HPLC (e.g., silica gel having pendant phenyl, butyl, or other hydrophobic groups) can be employed to further purify the protein. Some or all of the foregoing purification steps, in various combinations, are well known in the art and can be employed to provide an isolated and purified recombinant polypeptide.

Recombinant polypeptide produced in bacterial culture is usually isolated by initial disruption of the host cells, centrifugation, extraction from cell pellets if an insoluble polypeptide, or from the supernatant fluid if a soluble polypeptide, followed by one or more concentration, salting-out, ion exchange, affinity purification, or size exclusion chromatography steps. Finally, RP-HPLC can be employed for final purification steps. Microbial cells can be disrupted by any convenient method, including freeze-thaw cycling, sonication, mechanical disruption, or use of cell lysing agents.

### Agonists and Antagonists Screening

In another aspect, the present invention provides a screening method for identifying mast cell-expressed membrane protein agonists and antagonists. The screening method comprises exposing a mast cell-expressed membrane protein to a potential mast cell-expressed membrane protein agonist/antagonist and determining whether the potential agonist/antagonist interacts with the protein. If the potential agonist/antagonist interacts with the protein, particularly by binding to the protein, there is a strong presumption that the potential agonist/antagonist will actually function as an agonist or antagonist when administered in vivo to a patient and exposed to the native mast cell-expressed membrane protein. The agonists and antagonists identified using the method can be characterized as an agonist or an antagonist by exposing mast cells capable of producing mediators to the agonist/antagonist and measuring mast cell degranulation. Agonists will increase degranulation; antagonists will decrease degranulation. Another method for screening comprises transfecting the cells with a reporter gene constructs that contains MCEMP DNA binding sequences. Preferably, the potential agonist/antagonist is an organic compound or polypeptide, including antibodies. The screening methods are useful for identifying compounds that may function as drugs for preventing or treating diseases, particularly diseases characterized by relatively low or relatively high cytokine production compared to non-disease states.

### Adverse Side Effect Screening

In a further aspect, the present invention provides a screening method for determining whether pharmaceuticals are likely to cause undesirable side effects associated with reducing or increasing mast cell activity, particularly degranulation, when administered to an animal for the desired indication. The screening method comprises exposing mast cells expressing MCEMP or a purified MCEMP to the pharmaceutical and determining whether the pharmaceutical interacts with the protein or mimics the biological function of the protein ligand. If the pharmaceutical interacts with MCEMPs, there is a likelihood that the pharmaceutical will cause adverse side effects when administered to an animal for the desired indication. The adverse side effects result from an undesirable change in mast cell function or activity, particularly unwanted degranulation. Pharmaceuticals that can be screened by this method include, but are not limited to, polypeptides, proteins, peptides, glycoproteins, glycopeptides, glycolipids, polysaccharides, oligosaccharides, nucleotides, organic molecules, bioorganic molecules, peptidomimetics, pharmacological agents and their metabolites, and transcriptional and translation control sequences. In a preferred embodiment, antibodies to be administered for a particular indication are screened to see if they cross-react with MCEMPs and are therefore likely to cause unwanted side effects when administered for the intended indication.

### MCEMP Expression Modulation

In yet another aspect, the present invention provides a method for blocking or modulating the expression of MCEMPs by interfering with the transcription or translation of a DNA or RNA polynucleotide encoding the proteins. The method comprises exposing a cell capable of expressing MCEMPs to a molecule that interferes with the proper transcription or translation of a DNA or RNA polynucleotide encoding the protein. The molecule can be an organic molecule, a bioorganic molecule, an antisense nucleotide, a RNAi nucleotide, or a ribozyme.

In a preferred embodiment, the method comprises blocking or modulating the expression of MCEMPs by exposing a cell to a polynucleotide that is antisense to or forms a triple helix with MCEMP-encoding DNA or with DNA regulating expression of MCEMP-encoding DNA. The cell is exposed to antisense polynucleotide or triple helix-forming polynucleotide in an amount sufficient to inhibit or regulate expression of the proteins. Also, the present invention provides a method for blocking or modulating expression of MCEMPs in an animal by administering to the animal a polynucleotide that is antisense to or forms a triple helix with MCEMP-encoding DNA or with DNA regulating expression of MCEMP-encoding DNA. The animal is administered antisense polynucleotide or triple helix-forming polynucleotide in an amount sufficient to inhibit or regulate expression of MCEMPs in the animal. Preferably, the antisense polynucleotide or triple helix-forming polynucleotide is a DNA or RNA polynucleotide.

Methods for exposing cells to antisense polynucleotides and for administering antisense polynucleotides to animals are well known in the art. In a preferred method, the polynucleotide is incorporated into the cellular genome using know methods and allowed to be expressed inside the cell. The expressed antisense polynucleotide binds to polynucleotides coding for MCEMPs and interferes with their transcription or translation.

The methods are useful for inhibiting MCEMP expression while conducting research on various types of cells, e.g., mast cells or lung cells, and for preventing or treating animal disease characterized by excess cellular activity, particularly degranulation, compared to non-disease states.

### Disease Predisposition Diagnostic

In another aspect, the present invention provides a method for diagnosing the predisposition of a patient to develop diseases caused by unwanted activity of cells expressing MCEMPs. The invention is based upon the discovery that the presence of or increased amount of MCEMPs in certain patient cells, tissues, or body fluids indicates that the patient is predisposed to certain immune diseases. In one embodiment, the method comprises collecting a cell, tissue, or body fluid sample known to contain few if any MCEMPs from a patient, analyzing the tissue or body fluid for the presence of MCEMPs in the tissue, and predicting the predisposition of the patient to certain immune diseases based upon the presence of MCEMPs in the tissue or body fluid. In another embodiment, the method comprises collecting a cell, tissue, or body fluid sample known to contain a defined level of MCEMPs from a patient, analyzing the tissue or body fluid for the amount of MCEMPs in the tissue, and predicting the predisposition of the patient to certain immune diseases based upon the change in the amount of MCEMPs in the tissue or body fluid compared to a defined or tested level established for normal cell, tissue, or bodily fluid. The defined level of MCEMPs may be a known amount based upon literature values or may be determined in advance by measuring the amount in normal cell, tissue, or body fluids. Specifically, determination of MCEMPs levels in certain tissues or body fluids permits specific and early, preferably before disease occurs, detection of immune diseases in the patient. Immune diseases that can be diagnosed using the present method include, but are not limited to, the immune diseases described herein. In the preferred embodiment, the tissue or body fluid is mast cells and lung tissue.

### Disease Prevention and Treatment

In another aspect, the present invention provides a method for preventing or treating mast cell mediated diseases in a mammal. The method comprises administering a disease preventing or treating amount of a MCEMP agonist or antagonist to the mammal. The agonist or antagonist binds to MCEMP or its ligand and regulates the activity of the cell, particularly degranulation of mast cells, to produce mast cell mediator levels characteristic of non-disease states. Preferably, the disease is an allergy, asthma, autoimmune, or other inflammatory disease. Most preferably, the disease is an allergy or asthma.

The dosages of MCEMP agonist or antagonist vary according to the age, size, and character of the particular mammal and the disease. Skilled artisans can determine the dosages based upon these factors. The agonist or antagonist can be administered in treatment regimes consistent with the disease, e.g., a single or a few doses over a few days to ameliorate a disease state or periodic doses over an extended time to prevent allergy or asthma.

The agonists and antagonists can be administered to the mammal in any acceptable manner including by injection, using an implant, and the like. Injections and implants are preferred because they permit precise control of the timing and dosage levels used for administration. The agonists and antagonists are preferably administered parenterally. As used herein parenteral administration means by intravenous, intramuscular, or intraperitoneal injection, or by subcutaneous implant.

When administered by injection, the agonists and antagonists can be administered to the mammal in a injectable formulation containing any biocompatible and agonists and antagonists compatible carrier such as various vehicles, adjuvants, additives, and diluents. Aqueous vehicles such as water having no nonvolatile pyrogens, sterile water, and bacteriostatic water are also suitable to form injectable solutions. In addition to these forms of water, several other aqueous vehicles can be used. These include isotonic injection compositions that can be sterilized such as sodium chloride, Ringer's, dextrose, dextrose and sodium chloride, and lactated Ringer's. Nonaqueous vehicles such as cottonseed oil, sesame oil, or peanut oil and esters such as isopropyl myristate may also be used as solvent systems for the compositions. Additionally, various additives which enhance the stability, sterility, and isotonicity of the composition including antimicrobial preservatives, antioxidants, chelating agents, and buffers can be added. Any vehicle, diluent, or additive used would, however, have to be biocompatible and compatible with the agonists and antagonists according to the present invention.

### MCEMP Polypeptide Diagnostic

The antibodies of the present invention may also be used in a diagnostic method for detecting MCEMPs expressed in specific cells, tissues, or body fluids or their components. The method comprises exposing cells, tissues, or body fluids or their components to an antibody of the present invention that binds to a MCEMP and determining if the cells, tissues, or body fluids or their components bind to the antibody. Cells, tissues, or body fluids or their components that bind to the antibody cells, tissues, or body fluids or their components that bind to the antibody are diagnosed as cells, tissues, or body fluids that contain MCEMPs. Such method is useful for determining if a particular cell, tissue, or body fluid is one of a certain type of cell, tissue, or body fluid previously known to contain MCEMPs. Various diagnostic methods known in the art may be used, e.g., competitive binding assays, direct or indirect sandwich assays, and immunoprecipitation assays conducted in either heterogeneous or homogeneous phases.

Rather than using monoclonal antibodies in diagnostic kits, it may be possible to detect the presence of MCEMPs by using other compounds that bind to it, wherein the compounds are labeled and able to be detected. Such compounds may be isolated by screening compound libraries and/or peptide libraries. Library members, which are capable of interacting with MCEMPs, can be labeled with a fluorescent marker, or a radioactive marker, using a linker such as a peptide or other covalent chemical conjugate to join the compound with the marker. The resulting labeled compound can be used in a diagnostic kit to indicate the presence of MCEMP positive cells, using well-known methods.

### MCEMP Polypeptide Purification

The antibodies of the present invention may also be used in a method for isolating and purifying MCEMPs from recombinant cell cultures, contaminants, and native environments. The method comprises exposing a composition containing MCEMPs and contaminants to an antibody capable of binding to the MCEMPs, allowing the MCEMPs to bind to the antibody, separating the antibody-MCEMP complexes from the contaminants, and recovering the MCEMPs from the complexes. Various purification methods known in the art may be used, e.g., affinity purification methods that recover MCEMPs from recombinant cell culture or native sources. In this method, the antibodies that select MCEMPs are immobilized on a suitable support such a Sephadex resin or filter paper using methods well known in the art. The immobilized antibody then is contacted with a sample composition containing the MCEMPs to be purified and contaminants. The support is then washed with a suitable solvent capable of removing substantially all the material in the sample except the MCEMPs bound to the immobilized antibody. Finally, the support is washed with another suitable solvent that that removes the MCEMPs from the antibody.

### Knockout Animals

In another aspect, the present invention provides a knockout animal comprising a genome having a heterozygous or homozygous disruption in its endogenous MCEMP gene that suppresses or prevents the expression of biologically functional MCEMPs. Preferably, the knockout animal of the present invention has a homozygous disruption in its endogenous MCEMP gene. Preferably, the knockout animal of the present invention is a mouse. The knockout animal can be made easily using techniques known to skilled artisans. Gene disruption can be accomplished in several ways including introduction of a stop codon into any part of the polypeptide coding sequence that results in a biologically inactive polypeptide, introduction of a mutation into a promoter or other regulatory sequence that suppresses or prevents polypeptide expression, insertion of an exogenous sequence into the gene that inactivates the gene, and deletion of sequences from the gene.

Several techniques are available to introduce specific DNA sequences into the mammalian germ line and to achieve stable transmission of these sequences (transgenes) to each subsequent generation. The most commonly used technique is direct microinjection of DNA into the pronucleus of fertilized oocytes. Mice or other animals derived from these oocytes will be, at a frequency of about 10 to 20%, the transgenic founders that through breeding will give rise to the different transgenic mouse lines. Methods for generating transgenic animals via embryo manipulation and microinjection, particularly animals such as mice, have become conventional in the art, e.g., U.S. Pat. Nos. 4,736,866, 4,870,009, and 4,873,191 and in Hogan, B., Manipulating the Mouse Embryo, (Cold Spring Harbor Laboratory Press, Cold Spring Harbor, N.Y., 1986). Similar methods are used for production of other transgenic animals.

Embryonic stem cell (“ES cell”) technology can be used to create knockout mice (and other animals) with specifically deleted genes. Totipotent embryonic stem cells, which can be cultured in vitro and genetically modified, are aggregated with or microinjected into mouse embryos to produce a chimeric mouse that can transmit this genetic modification to its offspring. Through directed breeding, a mouse can thus be obtained that lacks this gene. Several other methods are available for the production of genetically modified animals, e.g., the intracytoplasmic sperm injection technique (ICSI) can be used for transgenic mouse production. This method requires microinjecting the head of a spermatocyte into the cytoplasm of an unfertilized oocyte, provoking fertilization of the oocyte, and subsequent activation of the appropriate cellular divisions of a preimplantation embryo. The mouse embryos thus obtained are transferred to a pseudopregnant receptor female. The female will give birth to a litter of mice. In ICSI applied to transgenic mouse production, a sperm or spermatocyte heads suspension is incubated with a solution containing the desired DNA molecules (transgene). These interact with the sperm that, once microinjected, act as a carrier vehicle for the foreign DNA. Once inside the oocyte, the DNA is integrated into the genome, giving rise to a transgenic mouse. This method renders higher yields (above 80%) of transgenic mice than those obtained to date using traditional pronuclear microinjection protocols.

### Vaccines

In another aspect, the present invention provides a vaccine useful for immunizing a mammal against mast cell or other MCEMP mediated diseases comprising a pharmaceutically acceptable carrier and one or more MCEMPs or immunogenic fragments thereof. The vaccine is administrated to mammals suffering from or susceptible to MCEMP mediated diseases. The vaccine induces the formation of antibodies in the immunized mammal that interact with MCEMPs and regulate the activity and function of cells expressing MCEMPs, including regulating the concentration of mast cells or other MCEMP expressing cells. The vaccine can contain one or more MCEMPs or immunogenic fragments alone or in combination with suitable adjuvants and/or other antigens and therapeutics.

In a further aspect, the present invention provides a method for immunizing a mammal against mast cell or other MCEMP mediated diseases comprising injecting one or more MCEMPs or immunogenic fragments thereof into the mammal. The MCEMP or immunogenic fragment can be injected alone or in combination with suitable adjuvants and/or other antigens and therapeutics.

Generally, antigens are presented to the immune system using major histocompatibility complex (MHC) molecules, i.e., MHC Class I molecules and MHC Class II molecules. Endogenous or self antigens, such as MCEMPs, are usually bound to MHC class I molecules and presented to cytotoxic T cells (“CTL(s)”). Exogenous antigens, such as viral antigens, are usually bound to MHC Class II molecules and presented to T cells that interact with B cells to produce antibodies.

Antigens presented via the Class II pathway, known as MHC Class II-restricted antigens or Class II antigens, are recognized by and activate T cells. These activated T cells cause a complete immune response to the Class II antigens. Because self antigens normally are not presented to the immune system through the MHC Class II pathway, the immune system does not recognize these self antigens as foreign and does not form a complete immune response to such antigens.

In one embodiment, a MCEMP antigen is injected in combination, simultaneously or contemporaneously, with other antigens that are designed to stimulate or manipulate the immune response. Preferably, the MCEMP antigen is injected as part of a construct comprising the MCEMP antigen and other antigens that are designed to induce a cellular immune response. Such other antigens are designed to enhance antigen presentation to T cells and induce a more potent immune response to antigens such as MCEMP that typically elicit an incomplete immune response because they are not recognized by the immune system as foreign antigens.

Typically, MCEMP is injected in combination with Class II antigens. Use of other antigens to stimulate the immune system via the MHC Class II pathway in combination with the MCEMP antigen, which may be recognized by the immune system as a self antigen that elicits a weak or incomplete immune response, helps to ensure that the MCEMP antigen will be treated by the immune system as a foreign antigen that elicits a complete immune system response. Preferably, the MCEMP antigen and the Class II antigen are part of a construct wherein the antigens are part of a single molecule. In another aspect, the present invention provides a construct comprising a MCEMP antigen and another antigen in a single molecule. Preferably, the other antigen is a Class II antigen.

In another aspect, the present invention provides a vaccine useful for immunizing a mammal against mast cell or other MCEMP mediated diseases comprising a pharmaceutically acceptable carrier and a vector containing a nucleic acid sequence encoding a MCEMP or antigenic fragment thereof. Preferably, the vaccine comprises a nucleotide sequence selected from the group consisting of SEQ ID NO:1; a variant of SEQ ID NO:1; and a fragment of SEQ ID NO:1. Most preferably, the vaccine comprises the nucleotide sequence that encodes the MCEMP having the sequence shown in SEQ ID NO:2 (“MCEMP1”) or antigenic fragment thereof, particularly an antigenic fragment comprising the extracellular domain (amino acids 106 through 187) or an antigenic fragment thereof.

The nucleotide vaccines of the present invention are useful for preventing or treating a disease caused by a malfunction of the immune system in distinguishing self from non-self. The vaccines cause the immune system to elicit self protective immunity and thus limit its own harmful activity to times when such a response is needed. In particular, DNA vaccines represent a novel means of expressing antigens in vivo for the generation of both humoral and cellular immune responses. This technology has proven successful in obtaining immunity not only to foreign antigens and tumors, but also to self antigens, such as a T cell receptor genes or autologous cytokines. Since DNA vaccines elicit both cellular and humoral responses against products of a given construct, the vaccines can be a very effective tool in eradicating diseased or unwanted cells. The direct injection of gene expression cassettes into a living host transforms a number of cells into factories for production of the introduced gene products. Expression of these delivered genes has important immunological consequences and may result in the specific immune activation of the host against the novel expressed antigens. This unique approach to immunization can overcome deficits of traditional antigen-based approaches and provide safe and effective prophylactic and therapeutic vaccines. The host normal cells (nonhemopoietic) can express and present MCEMP antigens to the immune system. The transfected cells display fragments of the antigens on their cell surfaces together with class I or class II major hisotcompatibility complexes (MHC I or MHC II). The MHC I display acts as a distress call for cell-mediated immune response, which dispatches CTLs that destroy the transfected cells. In general, when a cytopathic virus infects a host normal cell, the viral proteins are endogenously processed and presented on the cell surface, or in fragments by MHC molecules. Foreign defined nucleic acid transfected and expressed by normal cells can mimic viral infections.

An immunogenic fusion polypeptide encoded on a vector as described herein comprises a T cell epitope portion and a B cell epitope portion. A T cell epitope portion encoded on the vector comprises a broad range or “universal” helper T cell epitopes that bind the antigen presenting site of multiple (i.e., 2, 3, 4, 5, 6 or more) class II major histocompatibility (MHC) molecules and can form a tertiary complex with a T cell antigen receptor, i.e., MHC:antigen:T cell antigen receptor. A “non-endogenous protein” is a protein that is not endogenous to the mammal to be treated. Such non-endogenous proteins, or fragments thereof, useful as T cell epitope portions of the immunogenic fusion polypeptide include tetanus toxoid; diphtheria toxin; class II MHC-associated invariant chain; influenza hemagglutinin T cell epitope; keyhole limpet hemocyanin (KLH); a protein from known vaccines including pertussis vaccine, the Bacile Calmette-Guerin (BCG) tuberculosis vaccine, polio vaccine, measles vaccine, mumps vaccine, rubella vaccine, and purified protein derivative (PPD) of tuberculin; and also synthetic peptides which bind the antigen presenting site of multiple class II histocompatibility molecules, such as those containing natural amino acids described by Alexander et al. (Immunity, 1: 751-761 (1994)). When attached to a MCEMP epitope portion, the T cell epitope portion enables the immunogenic fusion polypeptide to break tolerance and permit antibodies to be made that react with endogenous MCEMPs. By “breaking tolerance” is meant forcing an organism to mount an immune response to a protein, such as endogenous MCEMPs, that the organism does not normally find immunogenic.

DNA vaccines recently have been shown to be a promising approach for immunization against a variety of infectious diseases. Michel, M L et al., Huygen, K, et al., and Wang, B, et al. Delivery of naked DNAs containing microbial antigen genes can induce antigen-specific immune responses in the host. The induction of antigen-specific immune responses using DNA-based vaccines has shown some promising effects. Wolff, J. A., et al. Recent studies have demonstrated the potential feasibility of immunization using a DNA-mediated vaccine for CEA and MUC-1. Conry, R. M., et al. and Graham, R. A., et al.

DNA-based vaccination has been shown to have a greater degree of control of antigen expression, toxicity, and pathogenicity than live attenuated virus immunization. The construction, operation, and use of the above pharmaceutically acceptable carriers for DNA vaccination and the above delivery vehicles are described in detail in U.S. Pat. No. 5,705,151 to Dow et al., entitled “Gene Therapy for T Cell Regulation”, which is directed at anti-cancer treatment, and is hereby incorporated by reference as if fully set forth herein.

In a further aspect, the present invention provides a method for immunizing a mammal against mast cell or other MCEMP mediated diseases comprising injecting a pharmaceutically acceptable carrier and a vector containing a nucleic acid sequence encoding a MCEMP or antigenic fragment thereof. Preferably, the method comprises injecting a vaccine comprising a nucleotide sequence selected from the group consisting of SEQ ID NO:1; a variant of SEQ ID NO:1; and a fragment of SEQ ID NO:1. Most preferably, the vaccine comprises the nucleotide sequence that encodes the MCEMP having the sequence shown in SEQ ID NO:2 (“MCEMP1”) or antigenic fragment thereof, particularly an antigenic fragment comprising the extracellular domain (amino acids 106 through 187) or an antigenic fragment thereof.

### Identification of MCEMP1

MCEMP1 was identified by subtractive hybridization using human mast cell mRNA as a tester and a combination of mRNAs from human THP-1 (˜45%), Daudi (˜35%) and TF-1 (˜20%) cell lines as drivers. Approximately 45 subtracted clones were isolated, sequenced, and used to search for matches in the publicly available nucleotide/protein databases. A cDNA clone comprising a 369 base pair (bp) insert isolated by the subtractive hybridization only matched to a number of EST clones that contain partial cDNA sequences, but it showed no significant homology to any cDNA sequences that encode known or predicted proteins in the GenBank database.

Two oligonucleotide primers:

| | |
|---|---|
| 5′ CTCCCAGAAAGGTGATGAA 3′ | (SEQ ID NO: 3) |
| and | |
| 5′ TAGACAGAAAACACGCCGCAGTA 3′ | (SEQ ID NO: 4) |

based on the 369 bp insert sequence were synthesized and used to screen a human peripheral blood leukocyte cDNA library (OriGene Technologies, Inc., Rockville, Md.). Several cDNA clones were isolated and sequenced. Three alternative splicing forms of mRNAs were identified by comparing the cDNA sequences with genomnic sequences in the GenBank database. Two of cDNA clones represent aberrant mRNA transcripts, because the putative translation product in all three reading frames would be aborted by stop codons. However, the majority of the cDNA clones predicted a protein product, which was derived from seven exons in the MCEMP1 gene. One such cDNA clone (9E) contained a full length coding region (564 bp) about 450 bp 5′ untranslated region and about 726 bp 3′ untranslated region.

In addition, a cDNA clone was obtained from a HMC-1 cell line by RT-PCR using an oligo primer covering the starting methionine codon 5′ GACCATGGAAGTGGAGGAAATCTAC 3′ (SEQ ID NO:5) and an oligo primer covering the stop codon, 5′ GCAGGTGCAGCCCCATCTT 3′ (SEQ ID NO:6). These cDNAs encoded a polypeptide of 187 amino acids. The predicted starting methionine codon was associated with a perfect Kozak sequence motif (ACCATGG), making it optimal for translation initiation. An allelic variation was found at amino acid residue 167 (Ile < >Val) among the cDNA clones, which was caused by a single nucleotide change at the first codon position (ATT < >GTT).

Computer-assisted analysis predicted that MCEMP1 had a transmembrane sequence located at amino acid residues 83 to 105. There did not appear to be a discernable N-terninal hydrophobic leader sequence. The predicted molecular mass for MCEMP1 was 21 kDa. A comparison of both nucleotide and amino acid sequences with GenBank or European Molecular Biology Laboratory databases revealed that it shared a 37% amino acid identity with BAB25183, a putative mouse sequence identified by The RIKEN Genome Exploration Research Group Phase II Team and the FANTOM Consortium. A 3-dimensional structure prediction was carried out using a threading-based fold recognition method (Kelley et al., J. Mol. Biol. 299:499-520 (2000)). Briefly, using a library of known protein structures, the MCEMP1 sequence was “threaded” and scored for compatibility. Four components were used in the scoring system: 1D and 3D sequence profiles coupled with secondary structure and salvation potential information. Since the prediction of transmembrane helix showed that MCEMP1 contained a transmembrane segment (amino acids 83 through 105), fold recognition process has been applied on the N-terminal part (amino acids 1 through 82) and C-terminal part (amino acids 106 through 187) separately to improve the accuracy. The results showed that the N-terminal region (amino acids 6 through 65) likely adopts an Ig-like β sandwich fold, sharing 21% identity with Ig domain of mouse T-cell receptor α-chain.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Experimental Methods

### Cell Culture

Human cord blood CD34⁺ cells (Bio-Whittaker, Walkersville, Md.) were cultured up to 9 weeks in culture media consisting of RPM11640 (Invitrogen) supplemented with 20% FBS (Sigma-Aldrich, St. Louis, Mo.), 2 mM L-glutamine, 50 μM 2-ME, 100 U/ml penicillin, 100 μg/ml streptomycin, 10 μg/ml gentamicin, 80 ng/ml SCF, 50 ng/ml IL-6 and 5 ng/ml IL-10. Cells were stained with anti-tryptase mAb to determine the percentage of mast cells. Cell suspensions were seeded at a density of 5×10⁵ cells/ml and cytokine-supplemented medium was replaced once a week. Recombinant human IgE was used for IgE cross-linking experiment. Other cell lines were cultured following ATCC's recommendations.

### Expression Construct and Transfection

Flag-tagged MCEMP1 cDNA was PCR-amplified by using two oligo primers:

| | |
|---|---|
| 5′ CACCATGGACTACAAAGACGATGACGACAAGGAAGTGGAGGAAATCTACAAGC 3′ | (SEQ ID NO: 7) |
| and | |
| 5′ TTGAGGTGAGGACTGTGGCATTT 3′. | (SEQ ID NO: 8) |

The PCR product was cloned into pcDNA3.1D/V5-His vector (Invitrogen). This yield the plasmid, MCEMP1-FV, that expresses MCEMP1 fusion protein with Flag tag at N terminus and V5 tag at C terminus. For the N-terminal region of MCEMP1 and Fc γ1 fusion construct (MCEMP1T-Fcγ1), the region encoding amino acid 1-83 was PCR-amplified, and joined to Fc γ1 coding region by additional round of PCR (SOEing, Ho, S. et al. 1989, Gene 77: 51-59). The coding region of the fusion protein was cloned into pSecTag/FRT/V5-His-TOPO (Invitrogen).
Transient transfection was performed using Lipofectamine Plus system (Invitrogen). Twenty micrograms of plasmid DNA was transfected into 293T cells in a 100 mm tissue culture dish; and 40 hours later, the cells were harvested in PBS-based, enzyme-free cell dissociation buffer (Invitrogen) for protein analysis.

### Protein Extraction and Western Blot Analysis

The whole cell protein sample was prepared by resuspending 3×10⁵ cells in 100 μl of ddH₂O, and heated at 98° C. for 5 minutes after adding equal volume of 2× sample loading buffer. To separate membrane fraction from soluble fraction, 5×10⁵ cells were subject to lysis procedure through either homogenization or freeze-thaw cycles. For homogenization, cells were first incubated in 150 μl of ddH₂O for 10 minutes, then passed through a #22 syringe needle multiple times. Thereafter one tenth of 10× lysis buffer (200 mM Tris-HCl, pH7.6; 700 mM KCl; 50 mM EDTA) was added back and incubated for 5 minutes. For the freeze-thaw method, cells were suspended in 1× lysis buffer, and freeze-thawed three times. Insoluble membrane fraction was separated from soluble proteins by centrifugation at maximum speed in a microcentrifuge.

The proteins were separated in a 15% SDS-PAGE. Western blot was performed as previously described [26] by using anti-Flag (Sigma) or anti-V5 mAb (Invitrogen).

### Immunofluorescence Staining

The transfected 293T cells (1×10⁶) were washed and preincubated at 4° C. for 20 minutes in 100 μl of the enzyme-free cell dissociation buffer (Invitrogen) containing 1% BSA. Cells were then incubated with FITC-conjugated anti-Flag (20 μg/ml) (Sigma-Aldrich) or Anti-V5 mAb (10 μg/ml) (Invitrogen) in the same buffer for 30 minutes. After three washes, cells were resuspended in 100 μl of 1×PBS with 1% paraformaldehyde. Alternatively, human cord blood-derived mast cells, HMC-1 and THP-1 cells were incubated with anti-MCEMP1 monoclonal antibodies, then incubated with 2^{nd} antibody, FITC-conjugated goat anti-mouse IgG antibody. All samples were analyzed using FACScan (Becton Dickinson, Franklin Lake, N.J.) and/or microscopy.

### Generation of Anti-MCEMP1 mAbs

Mice were immunized by antigen display constructs that contain the N (amino acid 1-83) and C terminal coding regions (amino acid 105-187). Hybridoma clones were generated and screened as conventional methods. In ELISA screening, we coated 96-well plate with either MCEMP1-FV or MCEMP1 fusion protein, then incubated with the supernatant of hybridoma clones. Goat anti-mouse IgG was used as 2^{nd} antibody to develop the signal.

### Immunoprecipitation of Biotinylated Membrane Protein

Cell surface membrane proteins were biotinylated in 10 mg/ml of D-biotinoyl-e-aminocaproic acid-N-hydroxysuccinimide ester (Boehringer)/10 mM sodium borate, pH8.8/150 mM sodium chloride. The cells were washed extensively, and lysed in 1×PBS with 0.5% NP40 and protease inhibitor mix (Boehringer). Immunoprecipitation was performed using anti-MCEMP1 mAb and Protein A-Sepharose beads (Amersham) following manufacturer's recommendations. The immunoprecipitated proteins were separated in SDS-PAGE and the biotinylated proteins were detected by streptavidin-HRP and ECL Detection Reagents (Amersham).

### Example 1

### Quantitative Real-time PCR Analysis of MCEMP1 mRNA Expression

Two sets of oligonucleotide primers:

| | |
|---|---|
| 5′ AAGGTGATGAATGAATAGGACTGA 3′ | (SEQ ID NO: 9) |
| and | |
| 5′ CCACCGTGACATGCCGAGACT 3′ | (SEQ ID NO: 10) |

were selected from the MCEMP1 nucleotide sequences using Primer Express 2.0 (Applied Biosystems, Inc.) and were synthesized and used in RT-PCR reactions to monitor the expression of MCEMP1.

Real-time quantitative PCR was performed with the ABI Prism 7900 (Applied Biosystems, Inc.) sequence detection system, using CYBR Green reagents, according to the manufacture's instructions. RNAs were isolated to measure the level of expression of MCEMP1 in the following cells: Daudi (a B lymphoblast cell line derived from Burkitt's lymphoma, ATCC No. CCL-213), THP-1 (a monocytic leukemia cell line, ATCC No. TIB202), TF-1 (a myeloid progenitor cell line, ATCC No. CRL-2003), HMC-1, (a mast cell line); primary monocytes; primary B cells; primary basophils; CD34+ progenitor cells; in vitro cultured cord blood derived mast cells (CBMC) at week 5 and week 9; macrophages and macrophages activated by LPS; HPB-ALL, (a T cell leukemia cell line); primary lymphocytes; neutrophils; and primary human vascular endothelial cells (HUVAC).

Equal amounts of each of the RNAs from the cell lines indicated above were used as PCR templates in reactions to obtain the threshold cycle (Cₜ). The Cₜ was normalized using the known Cₜ from 18S RNAs to obtain ΔCₜ. To compare relative levels of gene expression of MCEMP1 in different cell lines, ΔΔCₜ values were calculated by using the lowest expression level as the base, which were then converted to real fold expression difference values. MCEMP1 mRNA was found to be expressed in week 5 and week 9 in vitro cultured mast cells. Moderate levels were found in monocytes. Among the five human tissues examined, MCEMP1 was highly expressed in mast cells and lung cells but very little expression was observed in heart, liver, brain, trachea, and kidney.

### Example 2

### Expression of MCEMP1 Protein

To determine the MCEMP1 gene product, MCEMP1 cDNA was PCR-amplified by using two oligo primers:

| | |
|---|---|
| 5′ CACCATGGACTACAAAGACGATGACGACAAGGAAGTGGAGGAAATCTACAAGC 3′ | (SEQ ID NO: 11) |
| and | |
| 5′ TTGAGGTGAGGACTGTGGCATTT 3′ | (SEQ ID NO: 12) |

were cloned into pcDNA3.1D/V5-His vector (Invitrogen) with a Flag tag sequence attached to the N-terminus of MCEMP1 and a V5 tag fused to the C-terminus. The resultant clone, pMCEMP1-FV, was transiently transfected into 293T cells. Forty hours after transfection, transfected cells were harvested and separated into membrane and cytosolic fractions by either a homogenization or freeze-thaw method. Western blot analysis was performed using anti-Flag or anti-V5 mAb and anti-mouse IgG conjugates. MCEMP1 was expressed as a predominant 35 kDa protein. Minor forms of 29 and 32 kDa proteins were also present in MCEMP1 transfected cells. The fact that all the protein bands were larger than the calculated molecular weight, 27 kDa (21 kDa plus 6 kDa of tag), implies that MCEMP1 might be post-translationally modified, e.g., by glycosylation in 293T cells. Fractionation of cells resulted in the presence of MCEMP1 in the membrane fraction, but very little was present in the cytosol.

### Example 3

### Administering MCEMP1-binding Molecules

The antagonistic or agonistic MCEMP1 binding molecules, such as antibodies and biologically active fragments thereof, of the present invention can be administered to patients in an appropriate pharmacological formulation by a variety of routes, including, but not limited to, intravenous infusion, intravenous bolus injection, and intraperitoneal, intradermal, intramuscular, subcutaneous, intranasal, intratracheal, intraspinal, intracranial, and oral routes. Such administration enables them to bind to endogenous MCEMP1 and inhibit/stimulate the action MCEMP1. These antagonists can also block the binding of the natural ligand for MCEMP1.

The estimated dosage of such antibodies is between 10 and 500 μg/ml of serum. The actual dosage can be determined in clinical trials following the conventional methodology for determining optimal dosages, i.e., extrapolating a dosage range from in vitro and in vivo experiments, and then administering various dosages within the range to determine which is most effective.

### Example 4

### Subcellular Localization

To determine whether MCEMP1 is expressed as a type II transmembrane protein, MCEMP1 FV-transfected cells were lysed by either homogenization or freeze-thaw method and the membrane fraction (pellet) was separated from the soluble fraction by centrifugation. MCEMP1 was present mainly in the membrane fraction as detected by both anti-Flag and anti-V5 mAbs; very little was detected in soluble fraction. To further determine whether the Flag- and V5-tagged MCEMP1 is expressed on the cell surface and its orientation in the membrane, MCEMP1 FV-transfected cells were incubated in living condition with FITC-conjugated anti-Flag or anti-V5 mAb. Fluorescence microscopy and flow cytometric analysis showed that while anti-V5 mAb was bound to the MCEMP1-FV on the membrane, anti-Flag mAb did not bind to the membrane (Table 1). These results show that MCEMP1 is a type II transmembrane protein with the C-terminus exposed to the outside of the cellular membrane and the N-terminus to the cytoplasmic compartment.

### Example 5

### Characterization of Mouse Monoclonal Antibodies against MCEMP1

Mouse monoclonal antibodies (mAb) were generated against MCEMP1 and screened by ELISA, FACS, and Western blot analysis. Three of the mAb were characterized extensively. Antibody clone AZ1C11 bound to both MCEMP1-FV and MCEMP1T-Fcγ1, while antibody clones AZ1A8 and AZ3H6 only positively bound to full length of the MCEMP1 fusion protein. This shows that AZ1A8 and AZ3H6 specifically interact with C-terminal region of MCEMP1 and clone AZ1C11 interact with the N-terminal region of MCEMP1. Immuno-fluorescent staining of THP-1 and 293 T cells transfected with MCEMP1-Fc fusion protein confirmed the above results, i.e. antibody clones AZ1A8 and AZ3H6 bind to the C-terminal end of MCEMP1 in the living cells and clone AZ1C11 did not bind to the living cell. However, in Western blot analysis, AZ1A8 and AZ3H6 did not bind to MCEMP1 but clone AZ1C11 did bind to MCEMP1.

### Example 6

### Expression and Detection of Native MCEMP1 in Cord Blood-Derived Mast Cells (CBMC) and HMC-1 Cells

The real time RT-PCR analysis showed that MCEMP1 is differentially expressed in mast cells as well as in two long term-cultured cell lines, HMC-1 and THP-1. The immuno-fluorescent staining of CBMC, HMC-1, and THP-1 cells confirmed those results. The native MCEMP1 was detected by antibody clones AZ1A8 and AZ3H6 in those three types of cells but not detected in other cells tested. The binding of the antibodies to CBMC and HMC-1 cells behaved in a dose-dependent manner, i.e., the more antibody input resulted in bigger shift of the fluorescent intensity by the antibody-stained cells. The expression of MCEMP1 in CBMC was further assessed by immunoprecipitation of biotinylated membrane protein. A protein with molecular weight of ˜21 kD was detected by an anti-MCEMP1 antibody but not by any other antibodies tested. Because the detected MCEMP1 has the same molecular weight as predicted based on amino acid sequence, the native MCEMP1 is not glycosylated.

TABLE 1
Immuno-fluorescent staining of 293T
cells transfected with MCEMP1-FV

| | | |
|---|---|---|
| Antibody | Anti-V5 | Anti-Flag |

| Cell Status | Alive | Fixed | Alive | Fixed |
|---|---|---|---|---|
| pV252-FV | + | + | − | + |
| pcDNA3.1 | − | − | − | − |

In the specification, there have been disclosed typical prefefred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

1. A purified polypeptide comprising an amino sequence selected from the group consisting of:
SEQ ID NO:2;
a variant of SEQ ID NO:2;
a fragment of SEQ ID NO:2;
an amino acid sequence encoded by an isolated polynucleotide comprising a nucleotide sequence selected from the group consisting of:
SEQ ID NO:1;
a variant of SEQ ID NO:1; and
a fragment of SEQ ID NO:1.

2. The purified polypeptide of claim 1 wherein the polypeptide is encoded by a fragment of SEQ ID NO:1 comprising nucleotides 455 through 1018.

3. The purified polypeptide of claim 1 wherein the polypeptide is an agonist or antagonist that specifically binds to a mast cell-expressed membrane protein or its ligand and inhibits or activates the protein's cellular function.

4. The purified polypeptide of claim 3 wherein the polypeptide is an antagonist selected from the group consisting of soluble forms of mast cell-expressed membrane proteins and soluble polypeptides derived from the extracellular domains of mast cell-expressed membrane proteins that are capable of interfering with the ability of a mast cell-expressed membrane protein to interact with its natural ligand.

5. The purified polypeptide of claim 4 comprising an amino acid sequence selected from the group consisting of amino acids 106 to 187 of SEQ ID NO:2 or antagonist fragments thereof.

6. The purified polypeptide of claim 3 wherein the agonist or antagonist is an antibody.

7. The purified polypeptide of claim 6 wherein the antibody is selected from the group consisting of polyclonal, monoclonal, humanized, human, bispecific, and heteroconjugate antibodies.

8. The purified polypeptide of claim 6 wherein the antibody is a monoclonal antibody.

9. An isolated polynucleotde comprising a nucleotide sequence selected from the group consisting of:
SEQ ID NO:1;
a variant of SEQ I) NO:1;
a fragment of SEQ ID NO: 1;
a nucleotide sequence that encodes a polypeptide having the amino acid sequence selected from the group consisting of:
SEQ ID NO:2;
a variant of SEQ ID NO:2;
a fragment of SEQ ID NO:2.

10. The isolated polynucleotide of claim 9 comprising a nucleotide sequence that encodes a polypeptide having an amino acid sequence selected from the group consisting of amino acids 106 to 187 of SEQ ID NO:2 or fragments thereof.

11. An expression vector comprising the nucleotide sequence of claim 9.

12. An isolated host cell selected from the group consisting of a host cell comprising the expression vector of claim 11; a host cell comprising the nucleotide sequence of claim 9; and a host cell comprising the nucleotide sequence of claim 10.

13. A screening method for identifying mast cell-expressed membrane protein agonists and antagonists, comprising:
exposing a mast cell-expressed membrane protein to a potential mast cell-expressed membrane protein agonist antagonist; and
determining whether the potential agonist/antagonist interacts with the protein.

14. A screening method for determining whether pharmaceuticals are likely to cause undesirable side effects associated with reducing or increasing mast cell activity when administered to a mammal for the desired indication, comprising:
exposing mast cells expressing mast cell-expressed membrane proteins or a purified mast cell-expressed membrane protein to a pharmaceutical; and
determining whether the pharmaceutical interacts with the protein or mimics the biological function of the protein ligand.

15. A method for blocking or modulating the expression of a cellular mast cell-expressed membrane protein by interfering with the transcription or translation of a DNA or RNA polynucleotide encoding the mast cell-expressed membrane protein comprising exposing a cell capable of expressing a mast cell-expressed membrane protein to a molecule that interferes with the transcription or translation of a DNA or RNA polynucleotide encoding the mast cell-expressed membrane protein.

16. The method of claim 14 wherein the molecule is selected from the group consisting of antisense nucleotides, RNAi nucleotides, and ribozymes that interfere with the proper transcription or translation of a DNA or RNA polynucleotide encoding the mast cell-expressed membrane protein.

17. The method of claim 14 wherein the molecule is an antisense nucleotide that interferes with the proper transcription or translation of a DNA or RNA polynucleotide encoding the mast cell-expressed membrane protein.

18. A method for diagnosing the predisposition of a patient to develop diseases caused by unwanted activity of cells expressing mast cell-expressed membrane proteins, comprising:
collecting a cell, tissue, or body fluid sample known to contain few if any mast cell-expressed membrane proteins from a patient;
analyzing the tissue or body fluid for the presence of mast cell-expressed membrane proteins in the tissue; and
predicting the predisposition of the patient to certain immune diseases based upon the presence of mast cell-expressed membrane proteins in the tissue or body fluid.

19. The method of claim 18 wherein the cells expressing mast cell-expressed membrane proteins are mast cells and the unwanted activity is mast cell degranulation.

20. A method for diagnosing the predisposition of a patient to develop diseases caused by unwanted activity of cells expressing mast cell-expressed membrane proteins, comprising:
collecting a cell, tissue, or body fluid sample known to contain a defined level of mast cell-expressed membrane proteins from a patient;
analyzing the tissue or body fluid for the amount of mast cell-expressed membrane protein in the tissue; and
predicting the predisposition of the patient to certain immune diseases based upon the change in the amount of mast cell-expressed membrane protein in the tissue or body fluid compared to a defined or tested level established for normal cell, tissue, or bodily fluids.

21. The method of claim 20 wherein the cells expressing mast cell-expressed membrane proteins are mast cells and the unwanted activity is mast cell degranulation.

22. A method for preventing or treating mast cell-expressed membrane protein mediated diseases in a mammal comprising administering a disease preventing or treating amount of a mast cell-expressed membrane protein agonist or antagonist to the mammal.

23. The method of claim 22 wherein the mast cell-expressed membrane protein agonist or antagonist is an antibody.

24. A diagnostic method for detecting mast cell-expressed membrane proteins expressed in specific cells, tissues, or body fluids, comprising:
exposing cells, tissues, or body fluids or their components to an antibody that binds to a mast cell-expressed membrane protein; and
determining if the cells, tissues, or body fluids or their components bind to the antibody.

25. A method for producing an antibody that binds to mast cell-expressed membrane proteins, comprising a method selected from the group consisting of
using isolated mast cell-expressed membrane proteins or antigenic fragments thereof as an antigen;
using host cells that express recombinant mast cell-expressed membrane proteins as an antigen; and
using DNA expression vectors containing the mast cell-expressed membrane protein gene to express the mast cell-expressed membrane protein as an antigen for producing antibodies.

26. The antibody produced using the method of claim 25.

27. The antibody of claim 25 selected from the group consisting of polyclonal, monoclonal, humanized, human, bispecific, and heteroconjugate antibodies.

28. A method for isolating and purifying mast cell-expressed membrane proteins from recombinant cell culture, contaminants, and native environments, comprising:
exposing a composition containing mast cell-expressed membrane proteins and contaminants to an antibody capable of binding to the mast cell-expressed membrane proteins;
allowing the mast cell-expressed membrane proteins to bind to the antibody;
separating the antibody-mast cell-expressed membrane protein complexes from the contaminants; and
recovering the mast cell-expressed membrane proteins from the complexes.

29. The method of claim 28 wherein the antibody is an antibody of claim 25.

30. A transgenic knockout animal whose genome comprises a heterozygous or homozygous disruption in its endogenous mast cell-expressed membrane protein gene that suppresses or prevents the expression of biologically functional mast cell-expressed membrane proteins.

31. A vaccine useful for immunizing a mammal against mast cell or other mast cell-expressed membrane protein mediated diseases comprising a pharmaceutically acceptable carrier and one or more mast cell-expressed membrane proteins or immunogenic fragments thereof.

32. A method for immunizing a mammal against mast cell or other mast cell-expressed membrane protein mediated diseases comprising injecting one or more mast cell-expressed membrane proteins or immunogenic fragments thereof into the mammal.

33. A vaccine useful for immunizing a mammal against mast cell or other mast cell-expressed membrane protein mediated diseases comprising a pharmaceutically acceptable carrier and a vector containing a nucleic acid sequence encoding a mast cell-expressed membrane protein or antigenic fragment thereof.

34. The vaccine of claim 33 wherein the nucleic acid sequence is a sequence selected from the group consisting of SEQ ID NO:1; a variant of SEQ ID NO:1; and a fragment of SEQ ID NO:1.

35. A method for immunizing a mammal against mast cell or other mast cell-expressed membrane protein mediated diseases comprising injecting a pharmaceutically acceptable carrier and a vector containing a nucleic acid sequence encoding a mast cell-expressed membrane protein or antigenic fragment thereof.

36. The method of claim 35 wherein the nucleic acid sequence is a sequence selected from the group consisting of SEQ ID NO:1; a variant of SEQ ID NO:1; and a fragment of SEQ ID NO:1.


SEQUENCE LISTING (12 sequences)

SEQ ID NO: 1 - DNA, 1750 bp, Homo sapiens
ggggtggagctgaggggtggagctgaggctggagggaggaaggtgtggggggacccaggggtcctgtctccaagcctggttgctcttacgcgaaaagttgggacactgaggtgtcacagcttctcttttgaaatggagaggaggtaggagggtgaggtccatccaggtagacacagacacacacagagaccacagcttcctgtaacatttccgagtgtcgaattccatctcccggtctagaggtttttcttcttggtccttcctgagacctcttggctcccaagagcctcttgatcggggcaggaatgagggtgccccagggtgcgagagtcgtggatccctgaaaagaggaggctgctccccctctttcttccccccacctccagatttcctcatctgcccacaccttccggtgggcggggacgtgtatggacaaatttgcgggctggggaccatggaagtggaggaaatctacaagcaccaggaagtcaagatgcaagcaccagccttcagggacaagaaacagggggtctcagccaagaatcaaggtgcccatgacccagactatgagaatatcaccttggccttcaaaaatcaggaccatgcaaagggtggtcattcacgacccacgagccaagtcccagcccagtgcaggccgccctcagactccacccaggtcccctgctggttgtacagagccatcctgagcctgtacatcctcctggccctggcctttgtcctctgcatcatcctgtcagccttcatcatggtgaagaatgctgagatgtccaaggagctgctgggctttaaaagggagctttggaatgtctcaaactccgtacaagcatgcgaagagagacagaagagaggctgggattccgttcagcagagcatcaccatggtcaggagcaagattgatagattagagacgacattagcaggcataaaaaacgttgacacaaaggtacagaaaatcttggaggtgctgcagaaaatgccacagtcctcacctcaataaatgagaggacattgtggcagccaaagccacaacttggaagatggggctgcacctgccaacgaagacgggaaatgaccccccccccagcctagtgtgaacctgcccctcgtcccacgtatagaaaaacctcgagtcatggtgaatgagtgtctcggagttgctcgtgtgtgtgtacacctgcgtgcgtgtgtgtgcgtgtgtgcgcgtgtgttcgtgtgtgtgcgtgtgtgcgtgcgcgtgtgtgtgcattttgcaaagggtggacatttcagtgtatctcccagaaaggtgatgaatgaataggactgagagtcacagtgaatgtggcatgcatgcctgtgtcatgtgacatatgtgagtctcggcatgtcacggtgggtggctgtgtctgagcacctccagcagatgtcactctgagtgtgggtgttggtgacatgcattgcacgggcctgtctccctgtttgtgtaaacatactagagtatactgcggcgtgttttctgtctacccatgtcatggtgggggagatttatctccgtacatgtgggtgtcgccatgtgtgccctgtcactatctgtggctgggtgaacggctgtgtcattatgagtgtgccgagttatgccaccctgtgtgctcagggcacatgcacacagacatttatctctgcactcacattttgtgacttatgaagataaataaagtcaagggaaaaaaaaaaaaaaaaaaaaaaaaaa

SEQ ID NO: 2 - protein, 187 aa, Homo sapiens
MEVEEIYKHQEVKMQAPAFRDKKQGVSAKNQGAHDPDYENITLAFKNQDHAKGGHSRPTSQVPAQCRPPSDSTQVPCWLYRAILSLYILLALAFVLCIILSAFIMVKNAEMSKELLGFKRELWNVSNSVQACEERQKRGWDSVQQSITMVRSKIDRLETTLAGIKNVDTKVQKILEVLQKMPQSSPQ

SEQ ID NO: 3 - DNA, 19 bp, ARTIFICIAL
  Human Oligonucleotide sequence primer
ctcccagaaaggtgatgaa

SEQ ID NO: 4 - DNA, 23 bp, ARTIFICIAL
  Human Oligonucleotide sequence primer
tagacagaaaacacgccgcagta

SEQ ID NO: 5 - DNA, 25 bp, ARTIFICIAL
  Human Oligonucleotide Sequence Primer
gaccatggaagtggaggaaatctac

SEQ ID NO: 6 - DNA, 19 bp, ARTIFICIAL
  Human Oligonucleotide sequence primer
gcaggtgcagccccatctt

SEQ ID NO: 7 - DNA, 53 bp, ARTIFICIAL
  Human Oligonucleotide sequence primers
caccatggactacaaagacgatgacgacaaggaagtggaggaaatctacaagc

SEQ ID NO: 8 - DNA, 23 bp, ARTIFICIAL
  Human Oligonucleotide sequence primers
ttgaggtgaggactgtggcattt

SEQ ID NO: 9 - DNA, 24 bp, ARTIFICIAL
  Human Oligonucleotide sequence primer
aaggtgatgaatgaataggactga

SEQ ID NO: 10 - DNA, 21 bp, ARTIFICIAL
  Human Oligonucleotide sequence primer
ccaccgtgacatgccgagact

SEQ ID NO: 11 - DNA, 53 bp, ARTIFICIAL; identical to SEQ ID NO: 7
  Human Oligonucleotide sequence primer

SEQ ID NO: 12 - DNA, 23 bp, ARTIFICIAL
  Human Oligonucleotide sequence primer
ttgaggtgaggactgtggcattt